(19) United States
(12) Patent Application Publication
(10) Pub. No.: US 2025/0276174 A1
(43) Pub. Date: Sep. 4, 2025

(54) ELECTRODE LEAD ASSEMBLIES HAVING NERVE CUFFS AND NERVE CUFF STRAIGHTENERS

(71) Applicant: The Alfred E. Mann Foundation for Scientific Research, Valencia, CA (US)

(72) Inventor: Christopher Reed Jenney, Valencia, CA (US)

(73) Assignee: The Alfred E. Mann Foundation for Scientific Research, Valencia, CA (US)

(21) Appl. No.: 19/019,360

(22) Filed: Jan. 13, 2025

Related U.S. Application Data
(60) Provisional application No. 63/560,564, filed on Mar. 1, 2024.

(51) Int. Cl. A61N 1/05

(52) U.S. Cl. CPC A61N 1/0556

(57) ABSTRACT

An electrode lead assembly comprising a nerve cuff straightener and an electrode lead including a nerve cuff, associated with the distal end of the lead body, including a biologically compatible, elastic, electrically insulative cuff body that is configured to be circumferentially disposed around a nerve, has a pre-set furled state that defines an inner lumen and is movable to an unfurled state, and is configured to receive a portion of the nerve cuff straightener, and a plurality of electrically conductive contacts carried by the cuff body. When inserted into the nerve cuff, the portion of the nerve cuff straightener received by the nerve cuff moves the nerve cuff out of the pre-set furled state.

### CROSS-REFERENCE TO RELATED APPLICATION

This application claims the benefit of U.S. Provisional Application No. 63/560,564, filed Mar. 1, 2024, and entitled “Electrode Leads Assemblies Having Nerve Cuffs and Nerve Cuff Straighteners,” which is incorporated herein by reference.

### BACKGROUND OF THE INVENTIONS

### 1. Field of Inventions

The present inventions relate generally to the treatment of obstructive sleep apnea by stimulating the hypoglossal nerve.

### 2. Description of the Related Art

Obstructive sleep apnea (OSA) is a highly prevalent sleep disorder that is caused by the collapse of or increase in the resistance of the pharyngeal airway, often resulting from tongue obstruction. The obstruction of the upper airway is mainly caused by reduced genioglossus muscle activity during the deeper states of non-rapid eye movement (NREM) sleep. In some OSA patients, obstruction occurs predominantly during rapid eye movement (REM) sleep. This is known as REM OSA and has different cardiometabolic and neurocognitive risks. Obstruction of the upper airway causes breathing to pause during sleep. Cessation of breathing, in turn, causes a decrease in the blood oxygen saturation level, which is eventually corrected when the person wakes up and resumes breathing. The long-term effects of OSA include, but are not limited to, high blood pressure, heart failure, strokes, diabetes, headaches, and general daytime sleepiness and memory loss.

Some proposed methods of alleviating apneic events involve the use of neurostimulators to open the upper airway. Such therapy involves stimulating the nerve fascicles of the hypoglossal nerve (HGN) that innervate the intrinsic and extrinsic muscles of the tongue in a manner that prevents retraction of the tongue, which would otherwise close the upper airway during the inspiration portion of the respiratory cycle. In some instances, the trunk of the HGN is stimulated with a nerve cuff, including a cuff body and a plurality of electrically conductive contacts (sometimes referred to as “electrodes”) on the cuff body, that is positioned around the HGN trunk. To that end, some nerve cuffs are pre-shaped to a furled state, may assume slightly less furled states, and may be unfurled to a flattened state. The HGN trunk nerve cuff may be configured in such a manner that it can be used to selectively stimulate nerve fascicles which innervate muscles that extend the tongue, while avoiding other nerve fascicles, with what is predominantly radial vector stimulation. HGN branches may also be stimulated. For example, an HGN GM branch may be stimulated with what is predominantly axial vector stimulation.

Exemplary nerve cuffs are illustrated and described in U.S. Pat. Pub. Nos. 2018/0318577A1, 2018/0318578A1, 2019/0060646A1, 2019/0282805A1, 2022/0062629A1, 2022/0313987A1, 2023/0010510A1, 2023/0241394A1 and 2024/0009452A1, which are incorporated herein by reference in their entirety.

### SUMMARY

The present inventors have determined that nerve cuffs are susceptible to improvement. In particular, at least some nerve cuffs are pre-set (or “pre-shaped”) to a furled (or “curled”) state that causes the nerve cuff to self-wrap around the associated nerve. The present inventors have determined that nerve cuffs which are pre-set to a furled state are advantageous because, for example, they do not require the physician to manually wrap the cuff around the nerve and do not require sutures, or specialty clips or clamps, to hold the cuff is place, as do flat nerve cuffs. The present inventors have, nevertheless, determined that conventional nerve cuffs which are pre-set to a furled state are susceptible to improvement. For example, the present inventors have determined that the process of straightening the nerve cuff so that it may be placed around the nerve can be difficult, can damage the nerve cuff, can make it difficult to accurately place the nerve cuff, and can increase the duration of the surgical procedure.

An electrode lead assembly in accordance with at least one of the present inventions includes a nerve cuff straightener and an electrode lead including a nerve cuff, associated with the distal end of the lead body, including a biologically compatible, elastic, electrically insulative cuff body that is configured to be circumferentially disposed around a nerve, has a pre-set furled state that defines an inner lumen and is movable to an unfurled state, and is configured to receive a portion of the nerve cuff straightener, and a plurality of electrically conductive contacts carried by the cuff body. When inserted into the nerve cuff, the portion of the nerve cuff straightener received by the nerve cuff moves the nerve cuff out of the pre-set furled state. The present inventions also include systems with an implantable pulse generator or other implantable stimulation device in combination with such an electrode lead assembly.

There are several advantages associated with the present electrode lead assembly. By way of example, but not limitation, the physician can selectively adjust the curvature of the nerve cuff by moving the cuff straightener proximally and distally relative to the nerve cuff as the nerve cuff is being positioned around the HGN trunk or HGN GM branch. The nerve cuff will return to the pre-shaped furled state around the nerve when the stylet is removed therefrom. The ability to selectively unstraighten portions of the nerve cuff and adjust the curvature with the movable cuff straightener reduces the difficulty, duration and likelihood of cuff damage associated with the implantation process.

### BRIEF DESCRIPTION OF THE DRAWINGS

Detailed descriptions of exemplary embodiments will be made with reference to the accompanying drawings.

FIG. 1 is a plan view of a stimulation system including an electrode lead assembly in accordance with one embodiment of a present invention.

FIG. 2A is a side view of a portion of the stimulation system illustrated in FIG. 1.

FIG. 2B is an end view of a portion of the electrode lead assembly illustrated in FIG. 1.

FIG. 3 is a cut-away anatomical drawing of the head and neck area illustrating the muscles that control movement of the tongue, the HGN and its branches that innervate these muscles, and the nerve cuff illustrated in FIG. 1 on the HGN trunk.

FIG. 4 is a side view showing the nerve cuff illustrated in FIG. 1 in a pre-shaped furled state around a HGN branch.

FIG. 5 is a side view of the electrode lead assembly illustrated in FIG. 1 in a disassembled state.

FIG. 6 is a side view of the electrode lead assembly illustrated in FIG. 1 in an assembled state.

FIG. 7A is a section view taken along line 7A-7A in FIG. 5.

FIG. 7B is a section view of a portion of an electrode lead assembly in accordance with one embodiment of a present invention.

FIG. 7C is a side view of a portion of an electrode lead assembly in accordance with one embodiment of a present invention.

FIG. 8 is a front view of the nerve cuff illustrated in FIG. 1 in an unfurled state.

FIG. 9 is a rear view of the nerve cuff illustrated in FIG. 1 in an unfurled state.

FIG. 10 is a rear, cutaway view of the nerve cuff illustrated in FIG. 1 in an unfurled state.

FIG. 11 is a section view taken along line 11-11 in FIG. 10.

FIG. 12A is a section view of a portion of an electrode lead assembly in accordance with one embodiment of a present invention.

FIG. 12B is a section view of a portion of an electrode lead assembly in accordance with one embodiment of a present invention.

FIG. 13 is a front view of an electrode lead assembly in accordance with one embodiment of a present invention in an assembled state.

FIG. 14 is a rear, cutaway view of the electrode lead assembly illustrated in FIG. 13.

FIG. 15 is a rear, cutaway view of an electrode lead assembly in accordance with one embodiment of a present invention in an assembled state.

FIG. 16 is a block diagram of the stimulation system illustrated in FIG. 1.

### DETAILED DESCRIPTION OF THE EXEMPLARY EMBODIMENTS

The following is a detailed description of the best presently known modes of carrying out the inventions. This description is not to be taken in a limiting sense, but is made merely for the purpose of illustrating the general principles of the inventions.

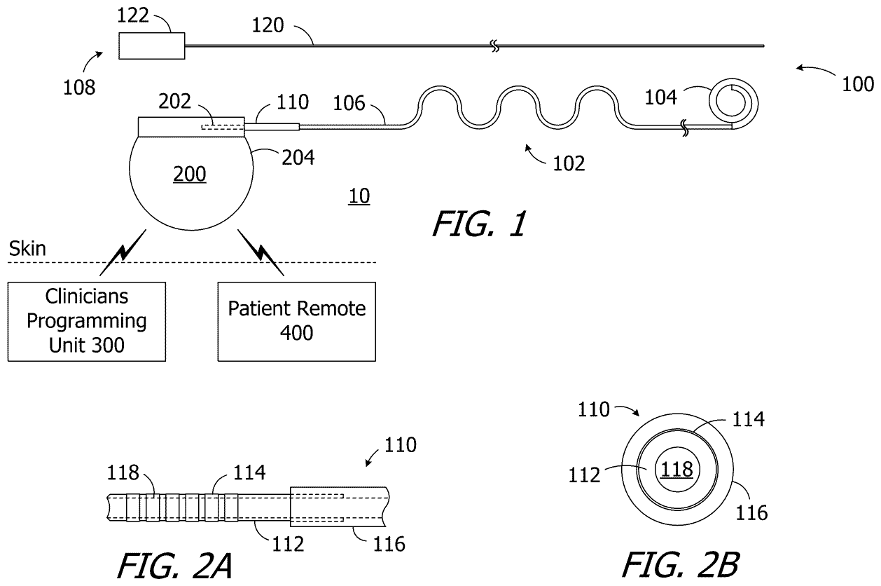

Referring to FIGS. 1-2B, a stimulation system 10 in accordance with one embodiment of a present invention includes an electrode lead assembly 100 and an implantable stimulator such as the implantable pulse generator (“IPG”) 200. A clinician's programming unit 300, a patient remote 400 and an IPG charger (not shown) may also be provided in some instances.

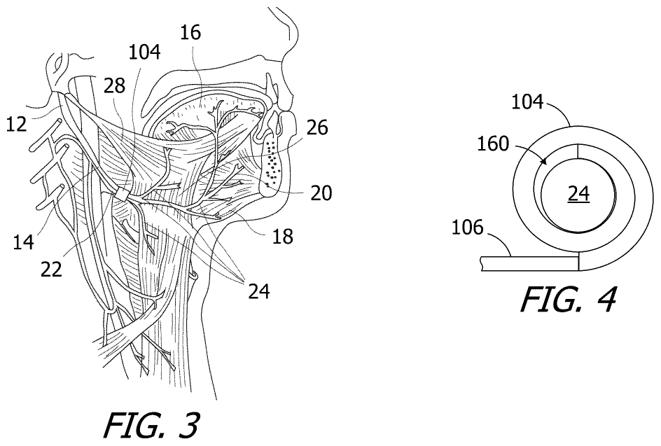

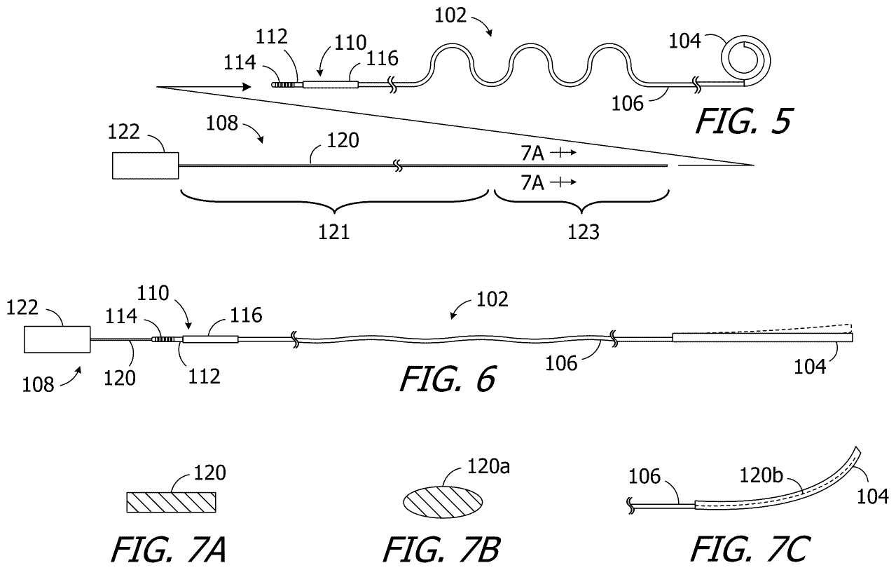

The exemplary electrode lead assembly 100 includes an electrode lead 102, with a nerve cuff 104 and a lead body 106, and a cuff straightener 108 that may be used to straighten the nerve cuff as it is placed onto and around a nerve in the manner described below. The lead body 106 couples the nerve cuff 104 to the IPG 200 by way of a lead connector 110, which has a connector body 112 and a plurality contacts 114, on the proximal end of the lead body 104. The IPG 200 has a corresponding connector receptacle 202. The lead connector 110 also includes a grip zone 116 and an internal lumen 118 that extends through the connector body 112 and the grip zone 116. The lumen 118 is configured to facilitate passage of a portion of the cuff straightener 108 in the manner described below with reference to FIGS. 5 and 6. The nerve cuff 104 is configured in such a manner that it may be circumferentially disposed around either the HGN trunk or a HGN branch (e.g., the HGN GM branch), as is described below with reference to FIGS. 3 and 4, and will assume a furled (or “curled”) state corresponding to the size of the HGN trunk or HGN branch when the cuff straightener 108 is removed therefrom.

The exemplary lead body 106 may include one or more S-shaped sections to provide strain relief (as shown) or may be straight. The S-shaped sections accommodate body movement at the location within the neck where the lead body 104 is implanted, thereby reducing the likelihood that the HGN will be damaged due to unavoidable pulling of the electrode lead 102 that may result from neck movements. The accommodation provided by the S-shaped sections also reduces the likelihood of fatigue damage. Additionally, although the exemplary system 10 includes a single electrode lead 102, other embodiments may include a pair of electrode leads 102 for bilateral HGN stimulation and an IPG (not shown) with two connector receptacles.

The exemplary cuff straightener 108 includes a stylet 120 and a handle 122. The stylet 120 may be inserted into and through the lead connector 110 and lead body 106 and into the nerve cuff 104 when the electrode lead 102 disconnected from the IPG 200 to straighten (or at least substantially straighten) the nerve cuff 104 is as described below with reference to FIGS. 5, 6 and 10. In other implementations, a cuff straightener may be inserted directly into a nerve cuff to straighten (or at least substantially straighten) the nerve cuff as is described below with reference to FIGS. 13 and 14.

Turning to FIG. 3, and as alluded to above, the nerve cuff 104 may be positioned around the trunk 14 of the HGN 12 and used to stimulate the muscles that anteriorly move the tongue 16 and, in particular, the fascicles of the HGN 12 that innervate the tongue protrusor muscles, such as the genioglossus 18 and/or the geniohyoid muscles 20. The nerve cuff 104 is positioned on the HGN trunk 14 at a position 22 proximal to the HGN branches 24. Although there are advantages to implanting the nerve cuff 104 at this proximal position 22, i.e., reduced surgical time and effort as well as reduced risk and trauma to the patient, it introduces the problem of inadvertently stimulating other fascicles of the HGN trunk 14 that innervate muscles in opposition to the genioglossus 18 and/or the geniohyoid muscles 20, i.e., the tongue retractor muscles, e.g., the hyoglossus 26 and styloglossus muscles 28, as well as the intrinsic muscles of the tongue 16. Accordingly, while some clinicians may desire to stimulate the HGN 12 at the HGN trunk 14, others may desire to stimulate the HGN at the GM branch 24. As illustrated in FIG. 4, the nerve cuff 104 is configured in such a manner that it may be positioned the HGN GM branch 24 as well as the trunk 14 and, in the illustrated implementation, the nerve cuff 104 is pre-set (or “pre-shaped”) to the furled (or “curled”) state illustrated in FIG. 4.

In one exemplary method of using the cuff straightener 108 to position the nerve cuff 104 of the electrode lead 102 around the HGN trunk 14 or HGN GM branch 24, the stylet 120 may be inserted though the connector 110 and lead body 106 until the distal end of the stylet reaches the distal region of the nerve cuff 104, as illustrated in FIGS. 5 and 6. The respective characteristics of the nerve cuff 104 and stylet 120 result in the nerve cuff transitioning to a straightened or at least substantially straightened state as shown in solid and dashed lines in FIG. 6. The insertion may be performed at the time of the implantation surgery, or when the electrode lead assembly 100 is manufactured, or at any other appropriate time. The cuff straightener 108 (and stylet 120) may be moved proximally and distally relative to the nerve cuff 104 as the nerve cuff is being positioned around the HGN trunk or HGN GM branch to selectively adjust the curvature of the nerve cuff 104. Portions of the nerve cuff 104 will return, either partially or completely, to the pre-shaped furled state as the stylet 120 is removed therefrom. Upon complete removal of the stylet 120, the nerve cuff 104 will return to a furled state, such as that illustrated in FIGS. 4 and 5, wrapped around the HGN trunk or HGN GM branch. The stylet 120 will also be removed from the remainder of the electrode lead 102 so that the lead connector 110 can be connected to the IPG 200.

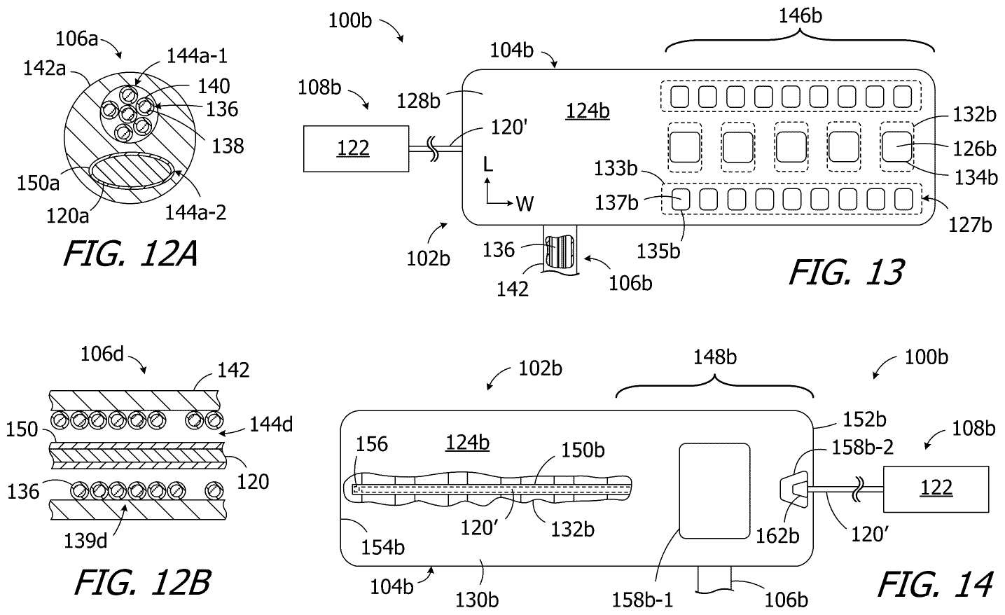

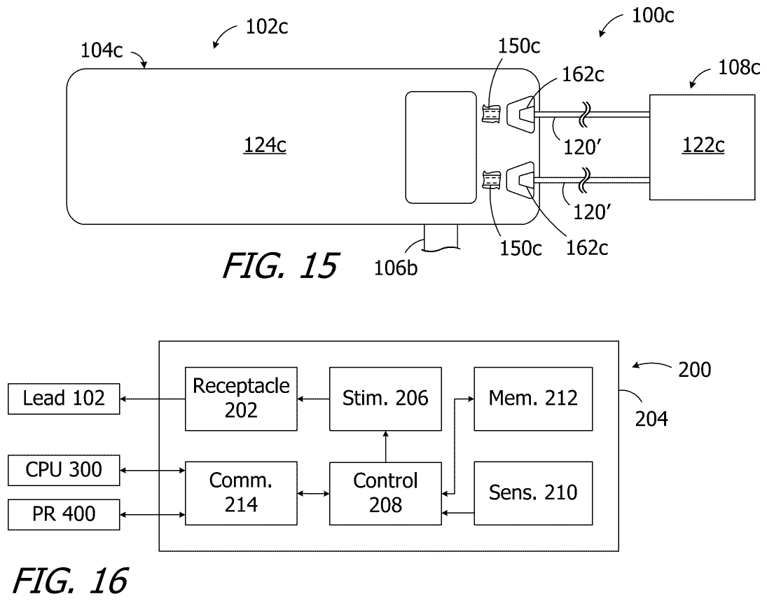

The exemplary cuff straightener 108 may include a wide variety of stylet materials and configurations to provide the desired cuff straightening characteristics such as stiffness and/or bending direction. Suitable materials include, but are not limited to 304 stainless steel, MP35N alloy, nitinol and tungsten. Exemplary cross-sectional shapes of the stylet 120 in a direction perpendicular to the longitudinal axis of the stylet include, but are not limited to, relatively flat ribbon-like shapes, rectangular shapes (FIG. 7A), rounded rectangular shapes (such as a flattened wire shape), and elliptical or otherwise oval shapes (e.g., stylet 120a in FIGS. 7B and 12). Stylets that do not include solid cross-sectional shapes, such as microcoils, may also be employed. Additionally, different portions of a particular stylet, such as the stylet 120 (and other stylets discussed herein), may have different stiffnesses, bending directions, cross-sectional shapes and sizes, and/or be formed from different materials (i.e., may have different straightening characteristics). The end portions, e.g., the distal-most 1 cm or more, of some stylets may be tapered. Referring for example to FIGS. 5 and 6, stylet region 121, which is located within the lead body 106 when the stylet is fully inserted into the electrode lead 102, may have different straightening characteristics than stylet region 123, which is located within the nerve cuff 104 and when the stylet is fully inserted into the electrode lead. There may also be more than two stylet regions with different characteristics. Stylets, such as stylet 120b in FIG. 7C, may also have straightening characteristics that allow the stylet to bend a nerve cuff, such as nerve cuff 104, into a particular curved shape, instead of straight or substantially straight, that is desirable for a particular implantation procedure. In still other implementations, electrode lead assemblies in accordance with the present inventions may include multiple stylets (as discussed below with reference to FIG. 15), or malleable stylets that allow the surgeon to bend portions of the stylets, and corresponding portions of the nerve cuff 104 and lead body 106, into desired shapes. As used herein, a “malleable” stylet is a stylet that can be readily bent by the physician to a desired shape, without springing back when released, so that it will remain in that shape during the implantation procedure.

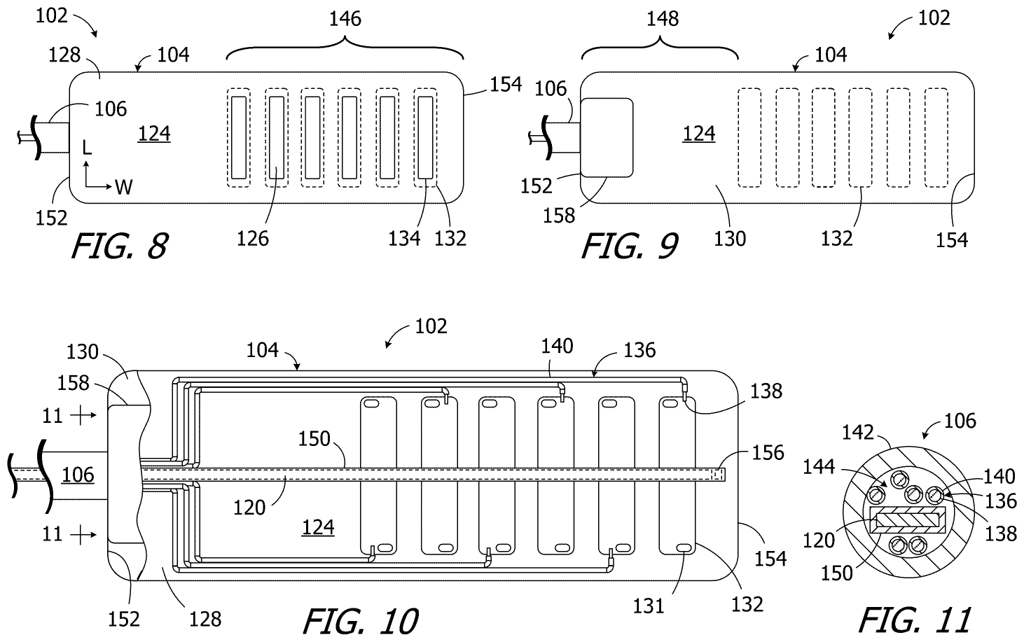

The present electrode leads may include any suitable nerve cuff configuration. As illustrated for example in FIGS. 8 and 9, the exemplary nerve cuff 104 includes a cuff body 124, which defines a length L and a width W that is greater than the length in the unfurled state, and a plurality of electrically conductive contacts (or “contacts”) 126 on the cuff body 124. Such contacts may also be referred to as “electrodes.” Although the present inventions are not so limited, the contacts 126 are narrow contacts, i.e., contacts with a greater length than width that extend in the length direction, and are spaced from one another in the width direction. Although the number may increase or decrease in the context of other nerve applications, there are six contacts 126 in the illustrated embodiment. With respect to shape, and although the present inventions are not so limited, the contacts 126 are the same shape, i.e., rectangles with rounded corners. The contacts 126 are also the same size. In other implementations, the contacts within a particular nerve cuff may differ in shape, size, and/or orientation. Other exemplary contact shapes include, but are not limited to, rounded rectangles, circles, ovals, and squares. Some or all of the contacts may also be relatively wide, as is discussed below with reference to FIGS. 13 and 14.

The cuff body 124 and contacts 126 may be of any suitable construction. In the illustrated implementation, the cuff body 124 includes a front layer 128 that will face the HGN trunk or branch and a rear layer 130 that will face away from the HGN trunk or branch. Conductive members 132 are located between the front layer 128 and rear layer 130 and may also include apertures 131 that, in conjunction with the material that forms the front and rear layers and enters the apertures, anchor the conductive members in their intended locations. The conductive members 132 are each exposed by way of respective openings 134 in the cuff body front layer 128. The openings 134 are located inwardly of the outer perimeter of the conductive members 132, which are shown in dashed lines in FIGS. 8 and 9. Referring to FIGS. 10 and 11, the contacts 126 in the illustrated embodiment may be individually electrically connected to the plurality contacts 114 on the lead connector 110 (FIG. 2) by wires 136 that extend through the lead body 106. Each wire 136 includes a conductor 138 and an insulator 140. The conductors 138 may be connected to the rear side of the conductive members 132 by welding or other suitable processes. The cables may be employed in place of the wires 136 in other implementations. In either case, to accommodate the wires or cables, the lead body 106 has an outer wall 142 that is annular in cross-section and an internal lumen 144 for the stylet 120 and wires 136.

With respect to the location of the contacts 126 in the exemplary electrode lead 102, and referring FIGS. 8-10, the cuff body 124 includes a stimulation region 146 and a compression region 148. The contacts 126 are located within the stimulation region 146. There are no contacts located within the compression region 148. The compression region 148 wraps around at least a portion of the stimulation region 146 when the nerve cuff 104 is in the pre-shaped furled state and slightly larger, expanded and less tightly furled states, thereby resisting (but not preventing) expansion of the stimulation region and improving the electrical connection between the contacts 126 and the HGN.

As noted above, the stylet 120 of the cuff straightener 108 may be inserted through the lead connector 110 and lead body 106 and into the nerve cuff 104 to straighten (or at least substantially straighten as shown in dashed lines in FIG. 6) the nerve cuff 104. To reduce the likelihood of damage to the nerve cuff 104 and/or to the lead body 106, the exemplary electrode lead 102 includes a tubular member 150, such as polymer tube or a metal coil, for the stylet 120. The tubular member 150, which also reduces friction, may be located only in the nerve cuff 104 or in both the nerve cuff 104 and the lead body 106. Referring to FIGS. 10 and 11, the tubular member 150 enters the cuff body 124 at cuff body proximal end 152 and terminates near cuff body distal end 154 and, accordingly, is coextensive with essentially the entire width W of the nerve cuff 104. The tubular member 150 also extends through the lead body 106 to the lead connector 110 and includes a stop 156 to prevent the stylet 120 from being pushed past the cuff body distal end 154. A strain relief 158 may be secured to the nerve cuff 104 and to the lead body 106 to reduce the likelihood of wire damage when pulling forces are applied to the lead body.

The exemplary cuff body 124 may be formed from any suitable material. Such materials may be biologically compatible, electrically insulative, elastic and capable of functioning in the manner described herein. By way of example, but not limitation, suitable cuff body materials include silicone, polyurethane and styrene-isobutylene-styrene (SIBS) elastomers. Suitable materials for the contacts 126 include, but are not limited to, platinum-iridium and palladium. The cuff materials (including the tubular member 150) should be pliable enough to allow the stylet 120 or a clinician to hold the cuff body 110 (and nerve cuff 104) in an unfurled state when the nerve cuff 104 is being placed around the HGN trunk (or HGN GM branch). The exemplary materials are also resilient enough to cause the nerve cuff return 104 to the pre-shaped furled state illustrated in FIG. 4 when the stylet 120 is removed, yet flexible enough to allow the cuff body 124 (and nerve cuff 104) to instead assume the slightly larger, expanded and less tightly furled states. For example, the inner lumen 160 defined by the cuff body 124 (and nerve cuff 104) in FIG. 4 is sized to accommodate an HGN structure that has a diameter of about 2.5 mm (e.g., the HGN GM branch 24). The cuff body 124 (and nerve cuff 104) is also cable of assuming less furled states to, for example, accommodate an HGN structure that has a diameter of about 3.0 mm (e.g., the HGN GM branch 24 in a swollen state) and an HGN structure that has a diameter of about 4.0 mm (e.g., the HGN trunk 22). The ability to assume slightly larger, expanded and less tightly furled states, in addition to the smaller fully furled state, allows the same nerve cuff 104 to accommodate either of the larger HGN trunk 14 or a smaller HGN branch 24. The ability to assume slightly larger, expanded furled states also allows the nerve cuff to accommodate nerve swelling that may occur post-surgery and to self-adjust to a smaller state when the swelling subsides. It should also be noted here that the width of the stimulation region 146 is such that it extends completely around the inner lumen 160, i.e., 360° or more around the longitudinal axis of the inner lumen, when the cuff body 124 is in the fully furled state illustrated in FIG. 4 that accommodates an HGN structure having a diameter of about 2.5 mm. The stimulation region 146 also extends substantially around the inner lumen 160, i.e., at least 288° in some examples and 360° or more in other examples, when the cuff body 124 is in an expanded and less tightly furled state that accommodates an HGN structure having a diameter of about 4.0 mm. The dimensions of the present nerve cuffs, including the various elements thereof, may by any dimensions that result in the nerve cuffs functioning as intended. With respect to the dimensions of the cuff body 124 of the exemplary nerve cuff 104, and referring to FIGS. 8 and 9, the cuff body is about 1.1 inches wide and about 0.34 inches long. As used herein in the context of dimensions, the word “about” means ±10-20%. The width of the stimulation region 146 is about 0.6 inches, while the width of the compression region 148 is about 0.5 inches.

Various aspects of the embodiments described above are susceptible to a wide variety of modifications. For example, lead bodies in accordance with the present inventions also may include separate lumens for the stylet 120 and the wires 136 in other implementations. To that end, and referring to FIG. 12A, the exemplary lead body 106a is a dual lumen extrusion that includes an outer wall 142a, an internal lumen 144a-1 for the wires 136, and an oval internal lumen 144a-2 for the oval stylet 120a and oval tubular member 150a. Referring to FIG. 12B, the wires 136 within an exemplary lead body 106d may be wound into a six-conductor helical coil 139d that abuts the inner surface of the outer wall 142 in other implementations. The coil 139d defines an internal lumen 144d, and the stylet 120 may extend though the internal lumen 144d. The stylet 120 may be located within a tubular member 150 (as shown) or the tubular member may be omitted.

Other modifications may relate to the manner in which stylets enter nerve cuffs and the configuration of the electrically conductive contacts on the nerve cuff. By way of example, but not limitation, the electrode lead assembly 100b illustrated in FIGS. 13 and 14 is similar to electrode lead assembly 100 and similar elements are represented by similar reference numerals. Here, however, the nerve cuff 104b of lead 102b includes first and second relatively wide contacts 127b that are spaced from one another in the length direction as well as a plurality of relatively narrow contacts 126b that are located between the relatively wide contacts 127b. As used herein, “relatively narrow” structures are structures that are shorter in the width direction than structures that are referred to as “relatively wide” and “relatively wide” structures are structures that are longer in the width direction than structures that are referred to as “relatively narrow.” Additionally, the straightener 108b may have a stylet 120′ that is relatively short (as compared to stylet 120) and that enters the nerve cuff 104b directly, as opposed to entering by way of the lead body 106b.

Referring first to the exemplary nerve cuff 104b and the associated contacts 126b and 127b, the cuff body 124b includes a front layer 128b and a rear layer 130b, and conductive members 132b and 133b are located between the front and rear layers. The conductive members 132b are each exposed by way of a respective opening 134b in the cuff body front layer 128b to define contacts 126b, while the conductive members 133b are each exposed by way of respective pluralities of openings 135b in the cuff body front layer 128b to define the exposed regions 137b of contacts 127b. The exposed regions 137b associated with each conductive member 133b together function as a single contact (i.e., one of the contacts 127b) because the exposed regions are part of the same conductive member. The contacts 126b and 127b are located within the stimulation region 146b and there are no contacts within the compression region 148b. The relatively narrow contacts 126b are centered relative to the relatively wide contacts 127b and are aligned with one another in the length direction in the illustrated implementation, but may be non-centered relative to the relatively wide contacts and/or offset from one another in the length direction in other implementations. The contacts 126b and 127b may be individually electrically connected to the plurality contacts 114 on the lead connector 110 (FIG. 2) by wires 136 in the manner described above with reference to FIG. 10. The wires 136 pass through the lead body 106b, and a strain relief 158b-1 may also be provided in some instances.

With respect to the accommodation of the stylet 120′, the exemplary nerve cuff 104b includes a tubular member 150b, such as a polymer tube or a metal coil. The tubular member 150b extends from, or is otherwise operably connected to, a port 162b near the cuff body proximal end 152b and terminates near cuff body distal end 154b. The port 162b provides direct access to the proximal end of the tubular member 150b. A stop 156 is provided at the distal end of the tubular member 150b. A strain relief 158b-2 may also be associated with the port 162b.

In still other embodiments, cuff straighteners may be provided with two or more stylets. To that end, the electrode lead assembly 100c illustrated in FIG. 15 is essentially identical to the electrode lead assembly 100b and similar elements are represented by similar reference numerals. For example, the exemplary electrode lead assembly 100c includes an electrode lead 102c, with a nerve cuff 104c and a lead body 106c, and a cuff straightener 108c. Here, however, the cuff straightener 108c includes two stylets 120′ and there are two tubular members 150c within the cuff body 124c. The two tubular members 150c are accessible by way of ports 162c. The cuff straightener 108c may include a single handle 122c that is connected to both stylets 120′ (as shown), or may include two separate handles.

The tubular member for the stylet in some embodiments may be removable. For example, the tubular member may be located on the exterior of rear side of the cuff body (as opposed to within the cuff body) and attached to the cuff body in such a manner that it can be easily removed during the implantation procedure.

It should also be noted here that the electrically conductive contact configuration illustrated in FIGS. 8-10 may be employed in a nerve cuff, such as that illustrated in FIGS. 13 and 14, where the stylet directly enters the nerve cuff, and that the electrically conductive contact configuration illustrated in FIGS. 13 and 14 may be employed in a nerve cuff, such as that illustrated in FIGS. 8-10, where the stylet directly enters the nerve cuff by way of the lead body. Other exemplary contact configurations that may be employed in either instance include, but are not limited to, those illustrated and described in U.S. Pat. Pub. Nos. 2018/0318577A1, 2018/0318578A1, 2019/0060646A1, 2019/0282805A1, 2022/0062629A1, 2022/0313987A1, 2023/0010510A1, 2023/0241394A1 and 2024/0009452A1.

Turning to FIG. 16, the exemplary IPG 200 includes the receptacle 202, a hermelically sealed outer case 204, and various circuitry (e.g., stimulation circuitry 206, control circuitry 208, sensing circuitry 210, memory 212, and communication circuitry 214) that is located within the outer case 204. The outer case 204 may be formed from an electrically conductive, biocompatible material such as titanium. The stimulation circuitry 206, which is coupled to the contacts 126 by way of the connector 110, receptacle 202 and wires 136, is configured to deliver stimulation energy to the HGN. The control circuitry 208 controls when and for how long the stimulation circuitry 206 applies stimulation, the intensity of the stimulation, the mode of stimulation (i.e., monopolar, bipolar or tripolar), and the particular contacts that are used in the stimulation. In the monopolar stimulation, at least a portion of the outer case 204 functions as a return electrode in the electrical circuit that also includes one or more of the contacts 126. In bipolar stimulation, the outer case 204 is not part of the electrical circuit and current instead flows from one of the contacts 126 to one of the other contacts 126. In tripolar stimulation, the outer case 204 is not part of the electrical circuit and current flows from one or more of the contacts 126 to more than one of the other contacts 126. The contacts that the current flows to form part of the return path for the stimulation energy, as do the associated wires connected thereto. The stimulation may also be predominantly axial vector stimulation, predominantly radial vector stimulation, or a hybrid of axial vector and radial vector.

It should also be noted here that in most instances, contacts that are entirely separated from (and electrically disconnected from) the associated nerve by the cuff body will not be used by the IPG for current transmission and return. For example, when the exemplary nerve cuff 104 is in a less lightly furled state and one of the contacts 126 is entirely separated from the GM branch 24 by the electrically non-conductive cuff body 124 and will not be used for current transmission or return. Such contacts may be identified by, for example, measuring the impedance at each contact.

The sensing circuitry 210 in the Illustrated embodiment may be connected to one or more sensors (not shown) that are contained within the outer case 204. Alternatively, or in addition, the sensors may be affixed to the exterior of the outer case 204 or positioned at a remote site within the body and coupled to the IPG 200 with a connecting lead. The sensing circuitry 210 can detect physiological artifacts that are caused by respiration (e.g., motion or ribcage movement), which are proxies for respiratory phases, such as inspiration and expiration or, if no movement occurs, to indicate when breathing stops. Suitable sensors include, but are not limited to, inertial sensors, bioimpedance sensors, pressure sensors, gyroscopes, ECG electrodes, temperature sensors, GPS sensors, and combinations thereof. The memory 212 stores data gathered by the sensing circuitry 210, programming instructions and stimulation parameters. The control circuitry 208 analyzes the sensed data to determine when stimulation should be delivered. The communication circuitry 214 is configured to wirelessly communicates with the clinician's programming unit 300 and patient remote 400 using radio frequency signals.

The control circuitry 208 may apply stimulation energy to either the HGN truck or an HGN branch (e.g. the HGN GM branch) in various stimulation methodologies by way of the cuff 104 when the patient is in the inspiratory phase of respiration, and other conditions for stimulation are met, thereby causing anterior displacement of the tongue to keep the upper airway unobstructed. The control circuitry 208 causes the stimulation circuitry 206 to apply stimulation in the form of a train of stimulation pulses during these inspiratory phases of the respiratory cycle (or slightly before the inspiration and ending at the end of inspiration) and not the remainder of the respiration cycle. The train of stimulus pulses may be set to a constant time duration or may change dynamically based on a predictive algorithm that determines the duration of the inspiratory phase of the respiratory cycle.

Although the inventions disclosed herein have been described in terms of the preferred embodiments above, numerous modifications and/or additions to the above-described preferred embodiments would be readily apparent to one skilled in the art. It is intended that the scope of the present inventions extend to all such modifications and/or additions. The inventions include any and all combinations of the elements from the various embodiments disclosed in the specification. The scope of the present inventions is limited solely by the claims set forth below.

1. An electrode lead assembly, comprising:
a nerve cuff straightener; and
an electrode lead including
an elongate lead body having a proximal end and a distal end, and
a nerve cuff, associated with the distal end of the lead body, including a biologically compatible, elastic, electrically insulative cuff body that is configured to be circumferentially disposed around a nerve, has a pre-set furled state that defines an inner lumen and is movable to an unfurled state, and is configured to receive a portion of the nerve cuff straightener, and a plurality of electrically conductive contacts carried by the cuff body;
wherein, when inserted into the nerve cuff, the portion of the nerve cuff straightener received by the nerve cuff moves the nerve cuff out of the pre-set furled state.

2. The electrode lead assembly claimed in claim 1, wherein
the nerve cuff includes a tubular member that is configured to receive the portion of the nerve cuff straightener.

3. The electrode lead assembly claimed in claim 1, further comprising:
a port on the cuff body configured to facilitate passage of the portion of the nerve cuff straightener into the cuff body.

4. The electrode lead assembly claimed in claim 3, wherein
the port is offset from the elongate lead body.

5. The electrode lead assembly claimed in claim 1, wherein
the elongate lead body is configured to facilitate passage of the portion of the nerve cuff straightener that is inserted into the nerve cuff from the proximal end of the lead body to nerve cuff.

6. The electrode lead assembly claimed in claim 5, wherein
the elongate lead body includes an internal lumen;
a lead connector, including an internal lumen, is on the proximal end of the elongate lead body; and
the elongate lead body internal lumen and the lead connector internal lumen are configured to facilitate passage of the portion of the nerve cuff straightener that is inserted into the nerve cuff.

7. The electrode lead assembly claimed in claim 5, wherein
the tubular member extends through the elongate lead body.

8. The electrode lead assembly claimed in claim 1, wherein
when inserted into the nerve cuff, the portion of the nerve cuff straightener moves the nerve cuff out of the pre-set furled state to an at least substantially straightened state.

9. The electrode lead assembly claimed in claim 1, wherein
when inserted into the nerve cuff, the portion of the nerve cuff straightener moves the nerve cuff out of the pre-set furled state to a curved shape.

10. The electrode lead assembly claimed in claim 1, wherein
the nerve cuff straightener includes a stylet; and
the portion of the nerve cuff straightener that is inserted into the nerve cuff comprises a portion of the stylet.

11. The electrode lead assembly claimed in claim 10, wherein
the stylet includes a first region with first straightening characteristics and a second region with second straightening characteristics that are different than the first straightening characteristics.

12. The electrode lead assembly claimed in claim 10, wherein
the stylet defines a longitudinal axis and one or more cross-sections in directions perpendicular to the longitudinal axis with shapes selected from group consisting of a flat ribbon-like shape, a rectangular shape, a rounded rectangular shapes), and an oval shape.

13. The electrode lead assembly claimed in claim 10, wherein
the stylet is malleable.

14. The electrode lead assembly claimed in claim 10, wherein
the stylet defines a distal end and a proximal end; and
the nerve cuff straightener includes a handle at the proximal end of the stylet.

15. The electrode lead assembly claimed in claim 1, wherein
the stylet comprises multiple stylets.

16. The electrode lead assembly claimed in claim 1, wherein
the cuff body defines a length, a length direction, a width in the unfurled state that is greater than the length, and a width direction; and
the electrically conductive contacts comprise relatively long contacts that are spaced from one another in the width direction.

17. The electrode lead assembly claimed in claim 1, wherein
the cuff body defines a length, a length direction, a width in the unfurled state that is greater than the length, and a width direction; and
the electrically conductive contacts comprise first and second relatively wide electrically conductive contacts that are spaced from one another in the length direction and extend in the width direction to such an extent that they extend completely around the cuff body inner lumen when the cuff body is in the pre-set furled shape and a plurality of relatively narrow electrically conductive contacts that are spaced from one another in the width direction and are located between the first and second relatively wide electrically conductive contacts.
